SEQUENCE LISTING (3 sequences)

SEQ ID NO: 1 - DNA, 2435 bp, Artificial Sequence
  Description of Artificial Sequence  Synthetic IncN plasmid R46
acgcgatatcgagctgataaaagggatccatcaggcaacgacgggctgctgccggccatcagcggacgcagggaggactttccgcaaccggccgttcgatgcggcaccgatggccttcgcgcaggggtagtgaatccgccaggattgacttgcgctgccctacctctcactagtgaggggcggcagcgcatcaagcggtgagcgcactccggcaccgccaactttcagcacatgcgtgtaaatcatcgtcgtagagacgtcggaatggccgagcagatcctgcacggttcgaatgtcgtaaccgctgcggagcaaggccgtcgcgaacgagtggcggagggtgtgcggtgtggcgggcttcgtgatgcctgcttgttctacggcacgtttgaaggcgcgctgaaaggtctggtcatacatgtgatggcgacgcacgacaccgctccgtggatcggtcgaatgcgtgtgctgcgcaaaaacccagaaccacggccaggaatgcccggcgcgcggatacttccgctcaagggcgtcgggaagcgcaacgccgctgcggccctcggcctggtccttcagccaccatgcccgtgcacgcgacagctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagccctgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagatttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagaaacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagcttgcatgccccttagggcagacctgcgtgaggcgggtgagagcaatattggtataatttttcagcaataag

SEQ ID NO: 2 - DNA, 792 bp, Artificial Sequence
  Description of Artificial Sequence  Synthetic portion of IncN plasmid R46
atgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacgtcggcttcccctggagagagcgagattctccgcgctgtagaagtcaccattgttgtgcacgacgacatcattccgtggcgttatccagctaagcgcgaactgcagtttggagaatggcagcgcaatgacattcttgcaggtatcttcgagccagccacgattgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcagaatcgcgccgaaggatgtcgctgccgactgggcaatggagcgcctgccggccagtatcagcccagtcatacttgaagctagacaggcttatcttggacaagaagaagatcgcttggcctcgcgcgcagatcagttggaagaatttgttcactacgtgaaaggcgagatcaccaaggtagtcggcaaataa

SEQ ID NO: 3 - protein, 263 aa, Artificial Sequence
  Description of Artificial Sequence  Synthetic aminoglycoside adenylyltransferase
MREAVIAEVSTQLSEVVGVIERHLEPTLLAVHLYGSAVDGGLKPHSDIDLLVTVTVRLDETTRRALINDLLETSASPGESEILRAVEVTIVVHDDIIPWRYPAKRELQFGEWQRNDILAGIFEPATIDIDLAILLTKAREHSVALVGPAAEELFDPVPEQDLFEALNETLTLWNSPPDWAGDERNVVLTLSRIWYSAVTGRIAPKDVAADWAMERLPASISPVILEARQAYLGQEEDRLASRADQLEEFVHYVKGEITKVVGK

(19) United States
(12) Patent Application Publication
(10) Pub. No.: US 2008/0113405 A1
(43) Pub. Date: May 15, 2008

(54) Gene-Cassette For Enhancement Of Protein Production

(75) Inventors: Sudeshna Kar, Gaithersburg, MD (US); Sankar L. Adhya, Gaithersburg, MD (US)

Correspondence Address: OFFICE OF TECHNOLOGY TRANSFER;NATIONAL INSTITUTES OF HEALTH, C/O HELLER EHRMAN WHITE & MCAULIFFE LLP, 1717 RHODE ISLAND AVENUE, NW, WASHINGTON, DC, 20036-3001, US

(73) Assignee: The Government of the United States of America as represented by the Secretary, Department of health, Rockville, MD (US)

(21) Appl. No.: 11/579,419

(22) PCT Filed: May 17, 2005
(86) PCT No.: PCT/US05/17001

§ 371 Date: Oct. 25, 2007

Related U.S. Application Data
(60) Provisional application No. 60/571,943, filed on May 18, 2004.

(51) Int. Cl. C12P 21/04; C12N 15/71; C12N 15/00; C12N 1/20

(52) U.S. Cl. 435/69.1; 435/471; 435/472; 435/474; 435/488; 435/320.1; 435/252.3; 435/252.33

(57) ABSTRACT

There are disclosed methods and compositions for gene expression and enhancement of protein production and/or accumulation. The invention provides gene-cassettes and methods of introducing the same into host cells for enhanced expression of target genes and production and/or accumulation of encoded proteins or peptides, or the like.

This application claims priority to U.S. provisional Ser. No. 60/571,943 filed May 18, 2004, the entirety of which is hereby incorporated by reference.

### BACKGROUND OF THE INVENTION

1. Field of the Invention

The invention relates to gene expression and enhancement of protein production and/or accumulation. More specifically, the present invention relates to gene-cassettes which confer antibiotic resistance and increased production of protein. The invention provides gene-cassettes and methods of introducing the same into host cells for enhanced expression of target genes, and production and/or accumulation of encoded proteins or peptides, or the like, and their use in biological systems.

2. Background of the Invention

The development of expression systems for production of recombinant proteins is important for developing a source of a given protein for research or therapeutic need. Gene expression systems have been developed for both prokaryotic cells, such as E. coli, and for eukaryotic cells, which includes both yeast (for example, Saccharomyces, Pichia and Kluyveromyces spp.) and mammalian cells. Gene expression in mammalian cells is often preferred for manufacturing of therapeutic proteins, since post-translational modifications in such expression systems are more likely to resemble those found in a mammal than the type of post-translational modifications that occur in microbial (prokaryotic) expression systems.

The bacterium Escherichia coli is one of the most commonly used prokaryotic host for production of heterologous recombinant proteins by expression and/or accumulation of the proteins intracellularly prior to extraction. To increase efficiency and lower costs of recombinant protein production, considerable efforts have been made to increase the amount of protein production per unit volume per unit time. Thus, there is a necessity in the art for development of a tool that would greatly increase the volumetric yield of recombinant proteins.

Several U.S. patents and research articles generally describe aspects of heterologous recombinant gene expression and recombinant protein production (see for example, U.S. Pat. Nos. 6,596,514, 6,271,207, 6,096,505, 5,658,763, and 5,089,397; Menzella et al. Biotechnol Bioeng. 2003, 82(7):809-17; Chao et al. Biotechnol Prog 2002, 18(2):394-400; and Bhandari et al. J. Bacteriol. 1997, 179(13):4403-6).

U.S. Pat. No. 6,596,514 discloses nucleotide sequences which can improve expression of recombinant proteins in eukaryotic cells from two- to eight-fold in stable cell pools when present in an expression vector. U.S. Pat. No. 6,271,207 describes methods of gene transfer to improve the expression of transgene up to 3-fold. U.S. Pat. No. 6,096,505 discloses methods for recombinant protein production by cotransfecting into a mammalian host cell three individual elements where they become operably linked such that expression of the selectable marker gene(s) necessarily requires coexpression of the gene of interest. U.S. Pat. No. 5,658,763 discloses methods for achieving enhanced protein production expressed from non-native gene constructs by transfecting DNA sequences to integrate into the genome. U.S. Pat. No. 5,089,397 describes an expression system for recombinant production of a desired protein using CHO cells transformed with a DNA sequence containing an operably linked enhancer capable of elevating the levels of production and/or a toxin-resistance conferring gene, which is capable of effecting amplification of the entire system.

Menzella et al. (Biotechnol Bioeng. 2003, 82(7):809-17) obtained recombinant protein production (up to 16 mg/L) by using genetically engineered a BL21 strain to allow the efficient use of lactose as inducer in fed-batch cultures. Chao et al. (Biotechnol Prog 2002, 18(2):394-400) discloses a high-level expression of heterologous genes in E. coli strain BL21 when constructed to carry a chromosomal copy of T7 gene 1.0 fused to the araBAD promoter. Bhandari et al. (J. Bacteriol. 1997, 179(13):4403-6) reported construction of a plasmid to obtain salt-induced overexpression of genes and overproduction of individual target gene products with NaCl as the inducer of T7 gene 1.0 in E. coli strain BL21.

However, none of the above were able to provide a system or a method that can enhance protein production, including recombinant proteins, by substantial amount. Moreover, such system can be physically or structurally unlinked to the target gene or the recombinant molecule for enhanced expression of a gene of interest (for example, a native or a recombinant molecule).

Therefore, there is a need in the art for development of a system or a method that would greatly increase expression of a target gene and enhance production of proteins, including recombinant proteins, using a standard, industrially-used host cell as well as other non-conventional hosts. The need is satisfied for the first time by the present invention.

### SUMMARY OF THE INVENTION

The present invention relates to gene expression/activation and enhancement of protein production and/or accumulation. The invention provides gene-cassettes and methods of introducing the same into host cells for enhanced expression of target genes and production and/or accumulation of encoded proteins or peptides or the like, and their use in biological systems.

In one aspect, the invention provides methods of enhancing the expression of a protein comprising: (a) transferring a gene-cassette into a host cell, wherein the gene-cassette comprises a polynucleotide of SEQ ID NO. 1 and/or SEQ ID NO. 2; and (b) culturing the cell under a suitable growth condition, thereby allowing production and/or accumulation of the protein.

In another aspect, the invention provides methods of reconstructing a host cell for production of a recombinant protein comprising: (a) transferring a gene-cassette into the host cell, wherein the gene-cassette comprises a polynucleotide of SEQ ID NO. 1 and/or SEQ ID NO. 2; (b) introducing a vector containing a recombinant gene for expression of the recombinant protein; and (c) culturing the host cell under a suitable growth condition, thereby allowing the production and/or accumulation of the recombinant protein.

In another aspect, the invention provides methods for enhancing production of a recombinant protein comprising: (a) transferring a gene-cassette into the host cell, wherein the gene-cassette comprises a polynucleotide of SEQ ID NO. 1 and/or SEQ ID NO. 2; (b) introducing a host cell a vector containing a recombinant gene for expression of the recombinant protein; and (c) culturing the cell under a suitable growth condition, thereby allowing the production and/or accumulation of the recombinant protein.

In another aspect, the invention provides a gene-cassette for enhanced production of a protein, wherein the gene-cassette comprises a polynucleotide of SEQ ID NO. 1 and/or SEQ ID NO.

Still in another aspect, the invention provides a gene-cassette, wherein the gene-cassette comprises a polynucleotide having at least 90 to 95% sequence identity to SEQ ID NO. 1 and/or SEQ ID NO. 2.

Yet in another aspect, the invention provides a gene-cassette, wherein the gene-cassette comprises a polynucleotide encoding a polypeptide having at least 90 to 95% sequence identity to SEQ ID NO. 3.

In a further aspect of the invention, a gene-cassette is inserted into the genome of a host cell. For example, in case of an E. coli cell, the cassette is inserted into yjaG gene or galR gene.

In another aspect, the gene-cassette is not physically or structurally linked to the vector carrying a gene for a recombinant protein, for example, the gene-cassette is not physically or structurally linked to a plasmid, a cosmid, a bacteriophage, or a virus.

Unless otherwise defined, all technical and scientific terms used herein in their various grammatical forms have the same meaning as commonly understood by one of ordinary skill in the art to which this invention belongs. Although methods and materials similar to those described herein can be used in the practice or testing of the present invention, the preferred methods and materials are described below. In case of conflict, the present specification, including definitions, will control. In addition, the materials, methods, and examples are illustrative only and are not limiting.

Further features, objects, and advantages of the present invention are apparent in the claims and the detailed description that follows. It should be understood, however, that the detailed description and the specific examples, while indicating preferred aspects of the invention, are given by way of illustration only, since various changes and modifications within the spirit and scope of the invention will become apparent to those skilled in the art from this detailed description.

### BRIEF DESCRIPTION OF THE DRAWINGS

FIG. 1 depicts two-dimensional protein gels of the total cellular proteins of wild-type strain MG1655 (FIG. 1A) and its derivative (recombinant) strain SK100 containing the 2.4 kb Tn21 cassette (FIG. 1B).

FIG. 2 is a photograph of a gel showing expression profile of recombinant protein 6His-MalE-HupA in wild-type BL21 and SK101, carrying the 6His-MalE-HupA clone in expression plasmid pExp66, after Ni-NTA column purification.

FIG. 3 depicts a graphical representation of total amount of recombinant 6His-MalE-HupA protein isolated from BL21 and SK101 strains in mg per liter of culture volume of equal cell contents.

FIG. 4 shows a photograph of a gel showing expression of 6-His-Carbonic anhydrase in wild-type BL21 and SK101, containing the anhydrase gene in vector pET15b, after Ni-NTA column purification.

FIG. 5 is a photograph of a gel showing expression of human anti-TAC VH protein in BL21 and SK101 strains, in crude cell lysates.

FIG. 6 shows time course of specific fluorescence intensity of GFPuv made from the pGFPuv plasmids introduced into BL21 and SK101 strains.

FIG. 7 is a gel photograph depicting total cellular protein profile of wild-type strain (MG1655), antibiotic-resistant recombinant strain containing the 2.4 kb Tn21 cassette in the yjaG gene in the chromosome (SK100), antibiotic-resistant recombinant strain containing only the aadA1 gene in the yjaG gene in the chromosome (SK102) and the antibiotic-resistant recombinant strain containing the aadA1 gene in galR gene in the chromosome (SK103).

FIG. 8 is a graphical representation of the total cellular protein content of wild-type and antibiotic-resistant recombinant strains in microgram/ml per 100 ml of culture volume containing about equal amounts of cells, as described in FIG. 7.

### DETAILED DESCRIPTION OF THE INVENTION

Definitions:

Expression Vector: The term “expression vector” refers to a plasmid, virus or other vehicle known in the art that has been manipulated by insertion or incorporation of the protein or polypeptide genetic sequences. This DNA element which renders the vector suitable for multiplication can be an origin of replication which works in procaryotic or eucaryotic cells. An example for an origin of replication which works in procaryotic cells is the colE1 ori. A recombinant vector further needs a selection marker for control of growth of these organisms. Suitable selection markers include genes which protect organisms from antibiotics (antibioticum resistance), for example, ampicillin, streptomycin, chloramphenicol or provide growth under compound deprived environmental conditions (auxotrophic growth conditions) when expressed as proteins in cells.

As used herein, the term “expression” refers to the biosynthesis of a gene product. For example, in the case of a structural gene, expression involves transcription of the structural gene into mRNA and the translation of mRNA into one or more polypeptides.

The term “cloning vector” refers to a nucleic acid molecule, for example, a plasmid, cosmid, or bacteriophage that has the capability of replicating autonomously in a host cell. Cloning vectors typically contain (i) one or a small number of restriction endonuclease recognition sites at which foreign DNA sequences can be inserted in a determinable fashion without loss of an essential biological function of the vector, and (ii) a marker gene that is suitable for use in the identification and selection of cells transformed or transfected with the cloning vector. Marker genes include genes that provide tetracycline resistance or ampicillin resistance, for example.

Expression of Recombinant Proteins: Recombinant expression vectors include synthetic or cDNA-derived DNA fragments encoding the protein, operably linked to suitable transcriptional or translational regulatory elements derived from mammalian, viral or insect genes. Such regulatory elements include a transcriptional promoter, a sequence encoding suitable mRNA ribosomal binding sites, and sequences which control the termination of transcription and translation, as described in the art. Mammalian expression vectors may also comprise nontranscribed elements such as an origin of replication, a suitable promoter and enhancer linked to the gene to be expressed, other 5′ or 3′ flanking nontranscribed sequences, 5′ or 3′ nontranslated sequences such as necessary ribosome binding sites, a polyadenylation site, splice donor and acceptor sites, and transcriptional termination sequences. An origin of replication that confers the ability to replicate in a host, and a selectable gene to facilitate recognition of transformants, may also be incorporated.

DNA regions are operably linked when they are functionally related to each other. For example, DNA for a signal peptide (secretory leader) is operably linked to DNA for a polypeptide if it is expressed as a precursor which participates in the secretion of the polypeptide; a promoter is operably linked to a coding sequence if it controls the transcription of the sequence; or a ribosome binding site is operably linked to a coding sequence if it is positioned so as to permit translation. Generally, operably linked means contiguous and, in the case of secretory leaders, contiguous and in reading frame. However, in case of the instant invention, the gene-cassette can be physically or structurally unlinked to a gene or a vector carrying a gene of interest and yet functionally related or operably linked by enhancing the expression of the gene or by enhancing the protein production.

The transcriptional and translational control sequences in expression vectors to be used in transforming vertebrate cells may be provided by viral sources. For example, commonly used promoters and enhancers are derived from Polyoma, Adenovirus 2, Simian Virus 40 (SV40), and human cytomegalovirus. Viral genomic promoters, control and/or signal sequences may be utilized to drive expression, provided such control sequences are compatible with the host cell chosen. Exemplary vectors can be constructed as disclosed by Okayama and Berg (Mol. Cell. Biol. 3:280, 1983). Non-viral cellular promoters can also be used (i.e., the β-globin and the EF-1 [symbol] promoters), depending on the cell type in which the recombinant protein is to be expressed.

DNA sequences derived from the SV40 viral genome, for example, SV40 origin, early and late promoter, enhancer, splice, and polyadenylation sites may be used to provide the other genetic elements required for expression of a heterologous DNA sequence. The early and late promoters are particularly useful because both are obtained easily from the virus as a fragment which also contains the SV40 viral origin of replication (Fiers et al., Nature 273:113, 1978). Smaller or larger SV40 fragments may also be used, provided the approximately 250 bp sequence extending from the Hind III site toward the BglI site located in the viral origin of replication is included.

The term “operably linked” is used to describe the connection between regulatory elements and a gene or its coding region. That is, gene expression is typically placed under the control of certain regulatory elements, including constitutive or inducible promoters, tissue-specific regulatory elements, and enhancers. Such a gene or coding region is said to be “operably linked to” or “operatively linked to” or “operably associated with” the regulatory elements, meaning that the gene or coding region is controlled or influenced by the regulatory element. However, a DNA fragment or a gene can be considered operably linked to another gene or a vector carrying a gene encoding a polypeptide in trans when they are functionally related to each other or enhance the polypeptide production.

Host Cells: Transformed host cells are cells which have been transformed or transfected with expression vectors constructed using recombinant DNA techniques and which contain sequences encoding recombinant proteins. Expressed proteins are preferably secreted into the culture supernatant, depending on the DNA selected, but may be deposited in the cell membrane.

Host cells may be cultured cells, explants, cells in vivo, and the like. Host cells may be prokaryotic cells, for example, E. coli, for example, strain BL21, or eukaryotic cells, for example, yeast, insect, amphibian, or mammalian cells, for example, Vero, CHO, HeLa, and others.

Various mammalian cell culture systems also can be employed to express recombinant protein. Examples of suitable mammalian host cell lines include the COS-7 lines of monkey kidney cells, described by Gluzman (Cell 23:175, 1981), and other cell lines capable of expressing an appropriate vector including, for example, CV-1/EBNA (ATCC CRL 10478), L cells, C127, 3T3, Chinese hamster ovary (CHO), HeLa and BHK cell lines.

A “cell line” refers to cultured cells that are immortal and can undergone passaging. Passaging refers to moving cultured cells from one culture chamber to another so that the cultured cells can be propagated to the subsequent generation.

A “recombinant host” may be any prokaryotic or eukaryotic cell that contains either a cloning vector or expression vector. This term also includes those prokaryotic or eukaryotic cells that have been genetically engineered to contain the cloned gene(s) in the chromosome or genome of the host cell.

Preparation of Host Cell and Transformation: Several Transformation protocols are known in the art, and are reviewed in Kaufman, R. J., Meth. Enzymology 185:537 (1988). The transformation protocol chosen is dependent on the host cell type and the nature of the gene of interest, and can be chosen based upon routine experimentation. The basic requirements of any such protocol are first to introduce DNA encoding the protein of interest into a suitable host cell, and then to identify and isolate host cells which have incorporated the heterologous DNA in a stable, expressible manner.

One commonly used method of introducing heterologous DNA is calcium phosphate precipitation, for example, as described by Wigler et al. (Proc. Natl. Acad. Sci. USA 77:3567, 1980). DNA introduced into a host cell by this method frequently undergoes rearrangement, making this procedure useful for cotransfection of independent genes.

Polyethylene-induced fusion of bacterial protoplasts with mammalian cells (Schaffner et al., Proc. Natl. Acad. Sci. USA 77:2163, 1980) is another useful method of introducing heterologous DNA. Protoplast fusion protocols frequently yield multiple copies of the plasmid DNA integrated into the mammalian host cell genome; however, this technique requires the selection and amplification marker to be on the same plasmid as the gene of interest.

A gene-cassette or a fragment of a gene can be introduced into the chromosome in the form of a linear DNA following a procedure as described in Yu et al. (Yu et al. Proc Natl Acad Sci USA. 2000, 97(11):5978-83).

Electroporation also can be used to introduce DNA directly into the cytoplasm of a host cell, for example, as described by Potter et al. (Proc. Natl. Acad. Sci. USA. 81:7161, 1988) or Shigekawa and Dower (BioTechniques 6:742, 1988). Unlike protoplast fusion, electroporation does not require the selection marker and the gene of interest to be on the same plasmid.

More recently, several reagents useful for introducing heterologous DNA into a mammalian cell have been described. These include Lipofectin® Reagent and Lipofectamine™ Reagent (Gibco BRL, Gaithersburg, Md.). Both of these reagents are commercially available reagents used to form lipid-nucleic acid complexes (or liposomes) which, when applied to cultured cells, facilitate uptake of the nucleic acid into the cells.

A method of amplifying the gene of interest is also desirable for expression of the recombinant protein, and typically involves the use of a selection marker (reviewed in Kaufman, R. J., supra). Resistance to cytotoxic drugs is the characteristic most frequently used as a selection marker, and can be the result of either a dominant trait (e.g., can be used independent of host cell type) or a recessive trait (e.g., useful in particular host cell types that are deficient in whatever activity is being selected for). Several amplifiable markers are suitable for use in the inventive expression vectors (for example, as described in Maniatis, Molecular Biology: A Laboratory Manual, Cold Spring Harbor Laboratory, NY, 1989; pages 16.9-16.14).

Useful regulatory elements, known in the art, also can be included in the plasmids used to transform mammalian cells. The transformation protocol chosen, and the elements selected for use therein, will depend on the type of host cell used. Those of skill in the art are aware of numerous different protocol and host cells, and can select an appropriate system for expression of a desired protein, based on the requirements of their cell culture systems.

The term “transformed cell” refers to a cell into which (or into predecessor or an ancestor of which) a nucleic acid molecule encoding a polypeptide of the invention has been introduced, by means of, for example, recombinant DNA techniques or viruses.

Transposon Tn21: Transposon Tn21 is a about 20 kb nucleic acid molecule (GenBank Locus: AF071413) (Nisen et. al. J. Mol. Biol. 117:975-998, 1977) found in plasmid NR1 (R100) and isolated from a Shigella flexneri strain (Nakaya, et al. Biochem. Biophys. Res. Commun. 3:654-659, 1960). The Tn21 is a subgroup of Tn3 family that contains closely related elements, which are largely responsible for multiple antibiotic-resistance in gram-negative bacteria. The Tn3 family of transposable elements is probably the most successful group of mobile DNA elements in bacteria: there are many different but related members and are widely distributed in gram-negative and gram-positive bacteria. Many transposons encoding multiple antibiotic-resistance in members of the family Enterobacteriaceae belong to the Tn21 subgroup. The transposon Tn21 is known to confer resistance to Streptomycin, spectinomycin, Sulfadimine, Mercury and Kanamycin (de la Cruz, F., and J. Grinsted. J. Bacteriol. 151:222-228, 1982). The transposon Tn21 and many of its closest relatives carry within them a potentially independently mobile DNA element called an integron. The integron encodes a RecA-independent, site-specific integration system that is responsible for the acquisition of multiple small mobile elements called gene cassettes that encode antibiotic-resistance genes.

Gene-cassette: The term “gene-cassette” refers to a cassette comprising a nucleic acid molecule (see for example, SEQ ID NO. 1), an aminoglycoside adenylyltransferase (aadA1) gene (see for example, SEQ ID NO. 2, GenBank Accession No. NC_{—}003292, Region: 36072-36863), a derivative of transposon Tn21 gene, a homologus molecule, or a fragment thereof, that can be introduced (for example, by transformation or electroporation) to a host cell (prokaryotic or eucaryotic) for enhanced expression of a target gene (for example, a native or a recombinant molecule) and enhanced production and/or accumulation of the encoded protein or polypeptide. The term “gene-cassette” also refers to a nucleic acid molecule that encodes a protein containing activity of aminoglycoside adenylyltransferase (see for example, SEQ ID NO. 3, GenBank Protein ID. NP_{—}511224.1 (aadA1)), a homologus molecule, or a fragment thereof. The “gene-cassette” also may confer resistance to aminoglycoside antibiotics, such as spectinomycin or streptomycin, to the host cell.

A “target gene”, as used herein, refers to an expressed gene in which modulation of the level of gene expression or of gene product activity enhance production and/or accumulation its encoded protein or polypeptide. A target gene can be a native gene of the host cell or a recombinant molecule introduced to the cell.

The term “gene”, in general, refers to a region on the genome that is capable of being transcribed to an RNA that either has a regulatory function, a catalytic function, and/or encodes a protein. An eukaryotic gene typically has introns and exons, which may organize to produce different RNA splice variants that encode alternative versions of a mature protein. The skilled artisan will appreciate that the present invention encompasses all target gene-encoding transcripts that may be found, including splice variants, allelic variants and transcripts that occur because of alternative promoter sites or alternative poly-adenylation sites. A “full-length” gene or RNA therefore encompasses any naturally occurring splice variants, allelic variants, other alternative transcripts, splice variants generated by recombinant technologies which bear the same function as the naturally occurring variants, and the resulting RNA molecules. A “fragment” of a gene, including an aadA1, can be any portion from the gene, which may or may not represent a functional domain, for example, a catalytic domain, a DNA binding domain, etc. A fragment may preferably include nucleotide sequences that encode for at least 25 contiguous amino acids, and preferably at least about 30, 40, 50, 60, 65, 70, 75 or more contiguous amino acids or any integer thereabout or therebetween.

The nucleic acid molecules of the invention, for example, the aadA1 gene or its subsequences, can be inserted into a vector, as described below, which will facilitate expression of a target gene. Accordingly, vectors containing the nucleic acids of the invention, cells transfected with these vectors, the polypeptides expressed, and antibodies generated against either the entire polypeptide or an antigenic fragment thereof, are among the aspects of the invention.

An “isolated DNA molecule” refers to a fragment of DNA that has been separated from the chromosomal or genomic DNA of an organism. Isolation also is defined to connote a degree of separation from original source or surroundings. For example, a cloned DNA molecule encoding an aadA1 gene is an isolated DNA molecule. Another example of an isolated DNA molecule is a chemically-synthesized DNA molecule, or enzymatically-produced cDNA, that is not integrated in the genomic DNA of an organism. Isolated DNA molecules can be subjected to procedures known in the art to remove contaminants such that the DNA molecule is considered purified, that is, towards a more homogeneous state.

An “isolated nucleic acid molecule” can refer to a nucleic acid molecule, depending upon the circumstance, that is separated from the 5′ and 3′ coding sequences of genes or gene fragments contiguous in the naturally occurring genome of an organism. The term “isolated nucleic acid molecule” also includes nucleic acid molecules which are not naturally occurring, for example, nucleic acid molecules created by recombinant DNA techniques.

The term “complementary DNA” (cDNA), often referred to as “copy DNA”, is a single-stranded DNA molecule that is formed from an mRNA template by the enzyme reverse transcriptase. Typically, a primer complementary to portions of the mRNA is employed for the initiation of reverse transcription. Those skilled in the art also use the term “cDNA” to refer to a double-stranded DNA molecule that comprises such a single-stranded DNA molecule and its complement DNA strand.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral methyl phosphonates, 2-O-methyl ribonucleotides, and peptide-nucleic acids (PNAs).

Unless otherwise indicated, a particular nucleic acid sequence also implicitly encompasses conservatively modified variants thereof (for example, degenerate codon substitutions) and complementary sequences, as well as the sequence explicitly indicated. Specifically, degenerate codon substitutions may be achieved by generating sequences in which the third position of one or more selected (or all) codons is substituted with suitable mixed base and/or deoxyinosine residues (Batzer et al., Nucleic Acid Res, 19:081, 1991; Ohtsuka et al., J. Biol. Chem., 260:2600-2608, 1985; Rossolini et al., Mol. Cell. Probes, 8:91-98, 1994). The term nucleic acid can be used interchangeably with gene, cDNA, mRNA, oligonucleotide, and polynucleotide.

The terms “sequence identity” in the context of two nucleic acid or polypeptide sequences includes reference to the residues in the two sequences which are the same when aligned for maximum correspondence over a specified comparison window, and can take into consideration additions, deletions and substitutions. When percentage of sequence identity is used in reference to proteins it is recognized that residue positions which are not identical often differ by conservative amino acid substitutions, where amino acid residues are substituted for other amino acid residues with similar chemical properties (for example, charge or hydrophobicity) and therefore do not deleteriously change the functional properties of the molecule.

Percentage of sequence identity, as described herein, means the value determined by comparing two optimally aligned sequences over a comparison window, wherein the portion of the polynucleotide sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “homologous” in their various grammatical forms in the context of polynucleotides means that a polynucleotide comprises a sequence that has a desired identity, for example, at least 60% identity, preferably at least 70% sequence identity, more preferably at least 80%, still more preferably at least 90% and even more preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, more preferably at least 70%, 80%, 90%, and even more preferably at least 95%.

Nucleotide sequences also can be substantially identical if two molecules hybridize to each other under stringent hybridization conditions. However, nucleic acids which do not hybridize to each other under stringent conditions are still substantially identical if the polypeptides which they encode are substantially identical. This may occur, for example, when a copy of a nucleic acid is created using the maximum codon degeneracy permitted by the genetic code. One indication that two nucleic acid sequences are substantially identical is that the polypeptide which the first nucleic acid encodes is immunologically cross reactive with the polypeptide encoded by the second nucleic acid, although such cross-reactivity is not required for two polypeptides to be deemed substantially identical.

An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Exemplary stringent hybridization conditions can be as following, for example: 50% formamide, 5×SSC and 1% SDS, incubating at 42° C., or 5×SSC and 1% SDS, incubating at 65° C., with wash in 0.2×SSC and 0.1% SDS at 65° C. Alternative conditions include, for example, conditions at least as stringent as hybridization at 68° C. for 20 hours, followed by washing in 2×SSC, 0.1% SDS, twice for 30 minutes at 55° C. and three times for 15 minutes at 60° C. Another alternative set of conditions is hybridization in 6×SSC at about 45° C., followed by one or more washes in 0.2×SSC, 0.1% SDS at 50-65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C. to 95° C. for 30 sec. to 2 min., an annealing phase lasting 30 sec. to 2 min., and an extension phase of about 72° C. for 1 to 2 min.

The terms “about” or “approximately” in the context of numerical values and ranges refers to values or ranges that approximate or are close to the recited values or ranges such that the invention can perform as intended, such as having a desired amount of nucleic acids or polypeptides in a reaction mixture, as is apparent to the skilled person from the teachings contained herein. This is due, at least in part, to the varying properties of nucleic acid compositions, age, race, gender, anatomical and physiological variations and the inexactitude of biological systems. Thus, these terms encompass values beyond those resulting from systematic error.

The present invention provides gene-cassettes which when transferred to host cells, for example, bacterial cells such as E. coli, induce enhancement of protein production and accumulation. More specifically, the gene-cassette (a derivative of transposon Tn21) confers resistance to aminoglycoside antibiotics such as spectinomycin and streptomycin. In addition, the gene-cassette causes increase in protein (cellular, native and/or recombinant) inside a recipient cell.

The gene-cassette, when transferred to a host cell, for example, a prokaryotic cell, such as E. coli strain BL21, increases the production of proteins by about 5 to about 200 fold or more.

The gene-cassette or a fragment of the gene can be introduced into the host cell chromosome in the form of a linear DNA following a procedure known in the art (for example, Yu et al. (Yu et al. Proc Natl Acad Sci USA. 2000, 97(11):5978-83). All subsequent insertions in the chromosomes also can be done following a similar method.

According to the invention, increased production of protein by the introduction of the gene-cassette is not restricted by the nature of host cell, vector, induction system, the nature of the protein, method used for introduction of the gene-cassette into the chromosome or the location of the gene cassette in the chromosome.

The invention also provides methods of reconstruction of host cells, such as a bacterial cell (E. coli, for example) for increased yield of protein.

The invention can be used to enhance efficacy, potency and immunogenicity of a live or an attenuated vaccine vector/strains by increasing the production of recombinant protective antigens/proteins through introduction of the gene-cassette (for example, a Tn21 gene-cassette) into the vaccine strain genome.

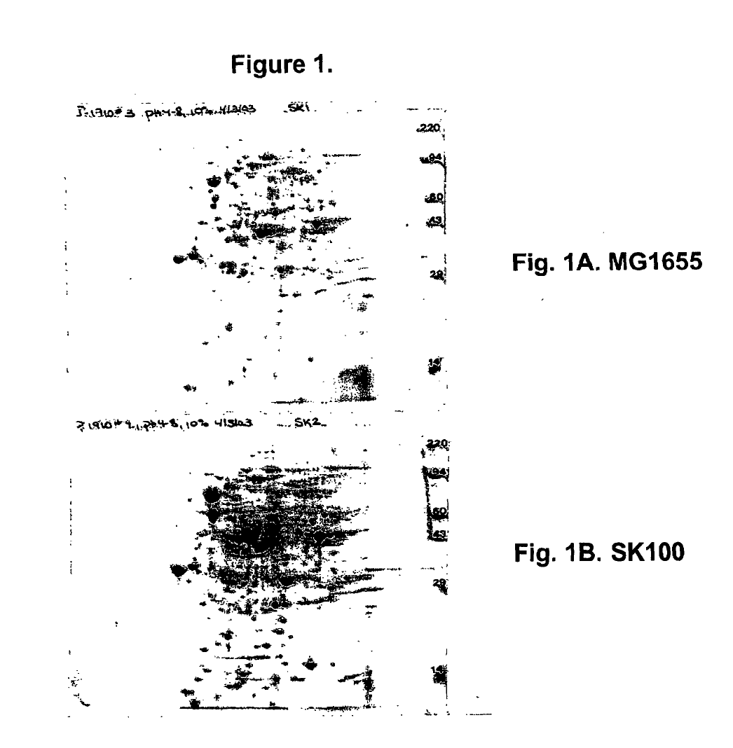

The gene-cassette, the methods, and/or the systems disclosed herein provide distinct advantages over the standard or conventional strains currently in use in the art. Advantages include enhanced product yield, reduced culture volume, faster processing and lower production cost. Results demonstrate dramatic increase in the expression level of proteins in gene-cassette carrying strains of the invention as opposed to the expression level observed in isogenic parental strains (see FIGS. 1-8). The gene-cassette, the methods, and/or the systems according to the instant invention, therefore, clearly offer advantages of increased productivity and lower processing time for successful implementation in pharmaceutical and biotechnological applications.

The invention provides gene-cassettes, which when transferred to E. coli strains induces activation/enhancement of native and/or recombinant protein production and/or accumulation. A 2.4 Kb DNA cassette, obtained from the transposon Tn21 (GenBank Accession No. NC_{—}003292), conferring resistance to aminoglycoside antibiotics, such as spectinomycin and streptomycin, was inserted in the yjaG gene of E. coli K12 strains (MG1655 and W3110) and E. coli B strain (BL21). Upon introduction of this gene-cassette, there was a sharp increase in the total cellular protein content in the cell. FIG. 1 shows a 2-D protein gel profiles from the parental strain (MG1655) and its derivative strain (SK100) containing the antibiotic resistance cassette. Extracts from equal amount of bacterial cells were analyzed for their total protein content. Estimation of the total protein by Bradford method coupled with the 2-D gel analysis indicated that there is a 5-10 fold increase in the total cellular protein content in SK100 over MG1655.

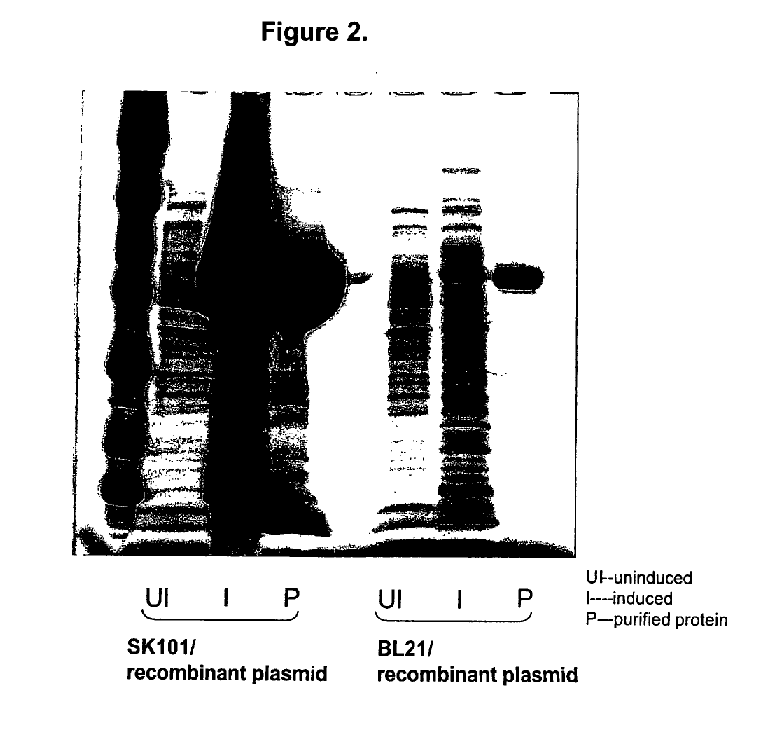

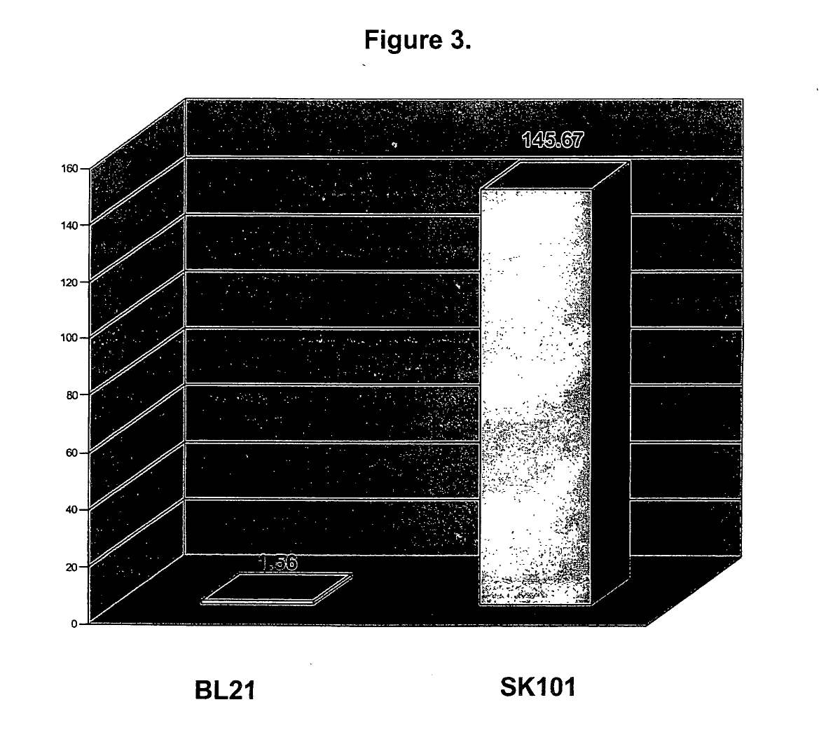

The effect of this gene-cassette on the expression of recombinant proteins in the cell was investigated. E. coli B, strain BL21, a commonly used industrial host for production of foreign proteins, was utilized along with its derivative strain (SK101), containing the antibiotic resistant cassette, for this purpose. A bacteriophage T7-based expression plasmid coding for a 6His-MalE-HupA fusion protein was chosen as a model recombinant protein to demonstrate the difference in expression level from the two strains. FIG. 2 illustrates the striking increase in the accumulation and yield of the recombinant protein, in both the cleared lysate as well as purified fraction, in SK101 over that in BL21. FIG. 3 depicts a quantitative estimation of the total volumetric yield of the recombinant protein from the two strains, SK101 and BL21. The yield from SK101 strain, after final purification, is about 140 fold higher than that from the BL21 strain. In most high-level expression systems, the maximum recombinant protein yield accounts for 20-30% of the total cell protein. In SK101, the recombinant protein yield was more than 60% of the total cell protein. Another notable feature was that, despite the tremendous increase in the final yield of the recombinant protein, the protein maintained its solubility and functional activity and did not aggregate into inactive inclusion bodies.

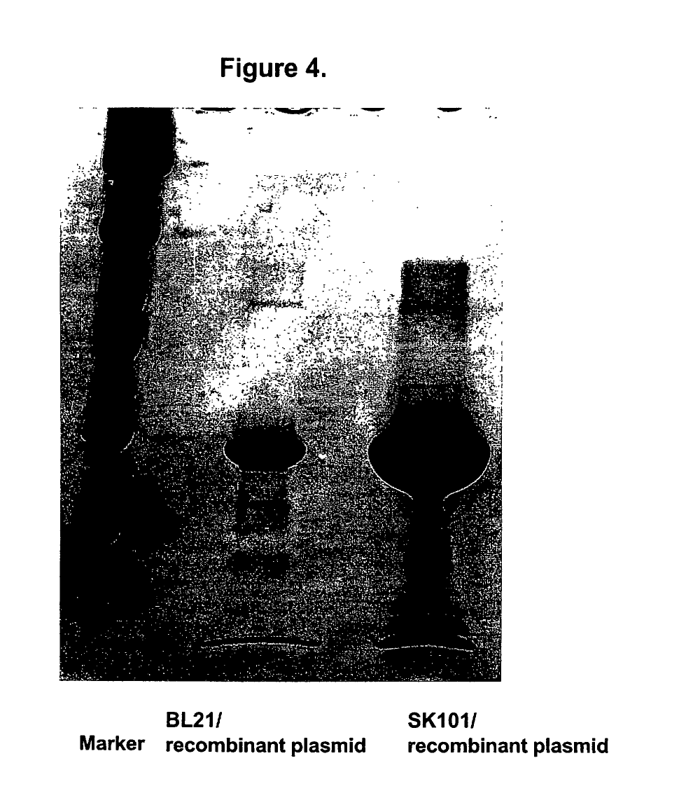

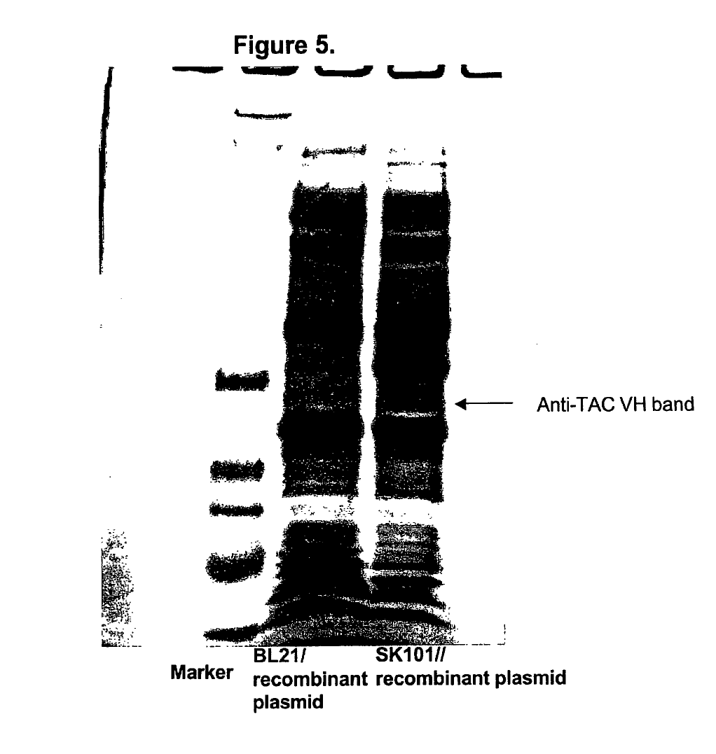

To demonstrate that the augmented protein yield is not specific to any particular promoter or recombinant gene, other expression plasmids containing different promoters and different recombinant genes also were tested. Two such examples are shown in FIGS. 4 and 5. FIG. 4 shows the total yield of E. coli carbonic anhydrase from SK101 and BL21 host cells. FIG. 5 shows the yield of human anti-TAC VH protein from the same two strains, SK101 and BL21. In each case, the protein yield was demonstrably higher in SK101, validating the potential universality of the gene-cassette or the system in enhancing recombinant protein production. In the case of human anti-TAC VH protein, there is little to no protein expression in BL21, manifesting the innate difficulty in expression of non-bacterial proteins in the systems currently in use. Therefore, the introduction of the cassette not only enormously increases the yield of the expressed proteins, but also alleviates the expression blocks in the synthesis of some refractory foreign proteins.

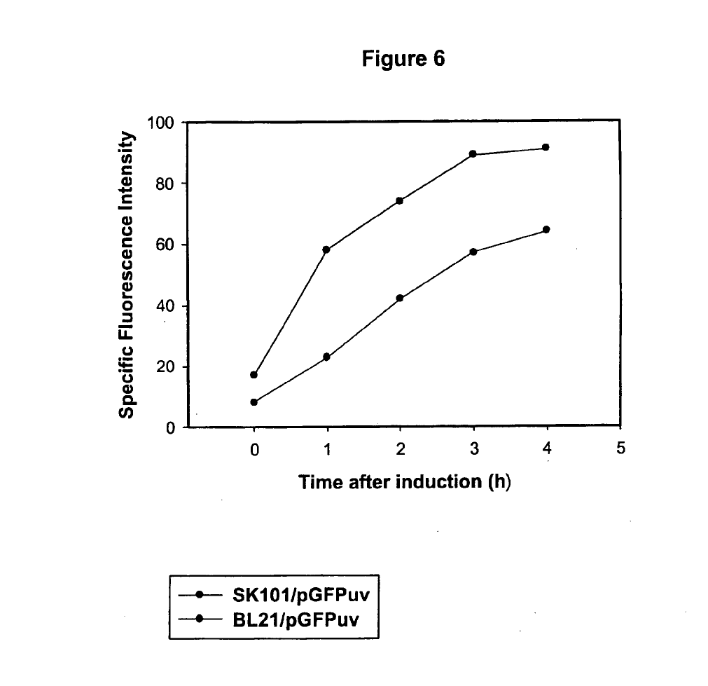

Apart from the final yield, the rate of synthesis of the recombinant protein also is critical for improving the productivity and lowering manufacturing costs. The effect of the gene-cassette system on the kinetics of recombinant protein production was investigated. FIG. 6 shows the specific rate of synthesis of green fluorescent protein (GFPuv) in BL21 and SK101 background. This investigation demonstrates that the rate of synthesis of foreign recombinant proteins in a system integrated with the gene-cassette also is faster than that in the conventional strain.

Although, most of the experiments were performed on derivatised strains of BL21, the gene-cassette also can be transferred to any host cell, including E. coli strains, by simple genetic techniques known in the art. Therefore, the application of the gene-cassette system is not limited by the choice of host, expression vector, or induction procedure.

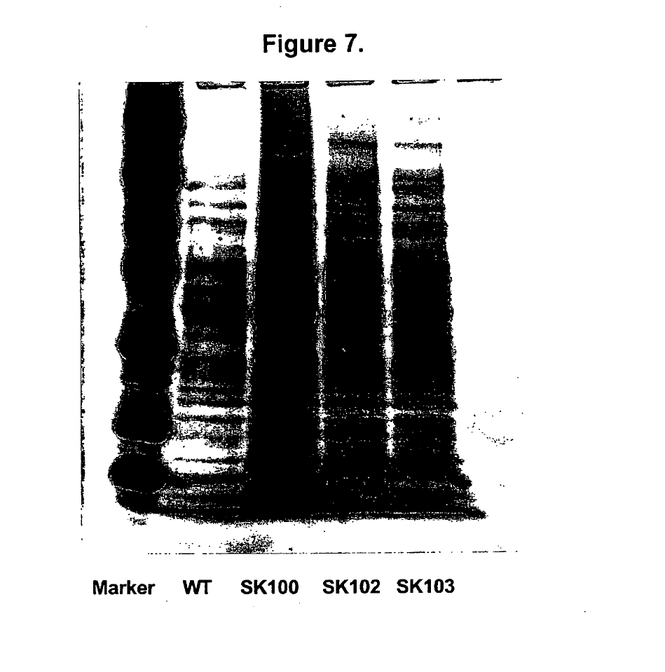

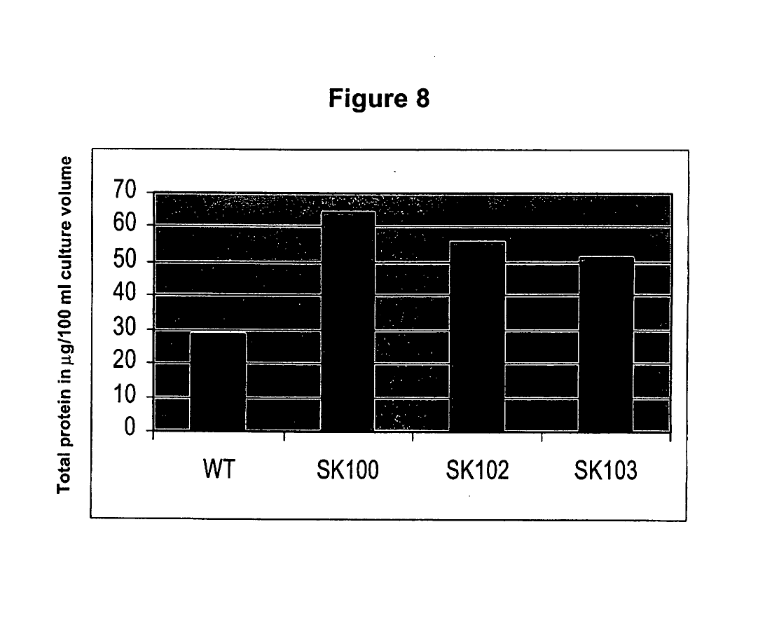

Further investigation of the gene-cassette revealed that a 792 bp aadA1 gene is primarily responsible for the increase in protein production. FIG. 7 shows the total cellular protein profiles of strains containing the entire 2.4 kb gene-cassette (SK100) (Lane 3 from left), the 792 bp aadA1 gene at the same position (on the yjaG gene) in the chromosome(SK102) (Lane 4 from left) and the aadA1 gene at a different position (on the galR gene) on the chromosome (SK103) (Lane 5 from left). Compared to the wild-type strain (MG1655) (Lane 2 from left) the total cellular protein content was higher in both the strains containing the entire 2.4 kb cassette (SK100) and the strain containing only the 792 bp aadA1 gene (SK102 and SK103). The strain containing the entire gene-cassette (SK100) produced slightly higher protein content than the strain containing only the 792 bp aadA1 gene (SK102 and SK103) (see FIG. 8). The results also show that the position of the aadA1 gene on the chromosome is not critical, as it had almost identical effects on the cellular protein content when inserted in the yjaG gene or in the galR gene (see FIG. 8).

The invention is further described by the following examples, which do not limit the invention in any manner.

### EXAMPLES

### Example I

### Gene-Cassette Induced Increase in Total Cellular Protein Content

A 2.4 Kb DNA cassette, obtained from the transposon Tn21 (see SEQ ID NO. 1), was inserted in the yjaG gene of E. coli K12 strains (MG1655) and E. coli B strain (BL21). E. coli cells were grown till mid-log phase (OD_{600 nm}-0.5) and total proteins were extracted from the cells. Proteins from cultures of equal OD were loaded on gels. Extracts from equal amount of bacterial cells were analyzed for their total protein content. Estimation of the total protein by Bradford method coupled with the 2-D gel analysis. The intensity of individual protein spots for majority of the proteins was greatly increased in the strain containing the 2.4 kb Tn21 cassette. The result indicates a 5-10 fold increase in the total cellular protein content in SK100 over MG1655 (see FIG. 1). Since the proteins loaded on the gels were taken from equal number of wild-type and recombinant cells, this experiment shows that most of the cellular proteins were expressed at elevated levels in the recombinant strain SK100. The 2-D protein gel profiles from the parental strain (MG1655) and its derivative strain (SK100) containing the antibiotic resistance cassette are shown in FIG. 1.

### Example II

### Effect of Gene-Cassette on the Expression of a Recombinant Protein

E. coli B, strain BL21, was used along with its derivative strain (SK101), containing the gene-cassette. A bacteriophage T7-based expression plasmid coding for a 6His-MalE-HupA fusion protein was chosen as a model recombinant protein to demonstrate the difference in expression level from the two strains. After transformation of BL21 and SK101 cells with the recombinant clone, cells were induced with 1 mM IPTG for 3 hours at 37° C. The expression of the recombinant protein was checked in the total cell lysate after induction (I) and also after purification on Ni-NTA columns (P). The results show a dramatic increase in the level of expression of the recombinant protein in SK101 strain containing the Tn21 cassette (see FIG. 2). A quantitative estimation of the total volumetric yield of the recombinant protein from the two strains (BL21 and SK101) indicates that the yield from SK101 is about 140 fold higher than that from BL21 (see FIG. 3).

### Example III

### Gene-Cassette Induced Enhanced Expression of 6-His-Carbonic Anhydrase

Production of E. coli carbonic anhydrase in SK101 and BL21 were determined. Recombinant protein production was induced by 1 mM IPTG for 3 hours at 37° C. The recombinant protein was purified using Ni-NTA columns before loading for gel electrophoresis. This experiment indicates an increased level of expression of 6-His-Carbonic anhydrase in the SK101 strain containing the Tn21 cassette (see FIG. 4). This experiment also demonstrates the universality of the gene-cassette in inducing enhanced expression of recombinant proteins, including 6-His-carbonic anhydrase and 6His-MalE-HupA, in the SK101 strain containing the Tn21 cassette (see FIGS. 3 and 4).

### Example IV

### Gene-Cassette Induced Enhanced Expression of Eukaryotic Protein

Expression of human anti-TAC VH protein was determined in BL21 and SK101 strains. The protein was induced by 1 mM IPTG for 3 hours and the whole cell lysates were loaded on gel to determine expression of the recombinant protein. The protein yield was demonstrably higher in SK101, validating the potential universality of the gene-cassette in inducing enhanced recombinant protein production. In the case of human anti-TAC VH protein, there is little to no protein expression in BL21 (see FIG. 5), manifesting the innate difficulty in expression of non-bacterial proteins in BL21, the strain currently in use industrially. Therefore, the introduction of the gene-cassette not only enormously increase the yield of the expressed proteins but also alleviates the expression blocks in the synthesis of some refractory foreign proteins. This experiment also demonstrates that the increased production of recombinant proteins in the gene-cassette carrying cells is not restricted to bacterial proteins, eukaryotic proteins also are expressed at higher levels.

### Example V

### Kinetics of Protein Production In Vivo

The effect of the gene-cassette system on the kinetics of recombinant protein production was investigated in BL21 and SK101 strains transformed with pGFPuv plasmids. The specific rate of synthesis of green fluorescent protein (GFPuv) in BL21 and SK101 background was determined. This experimental results indicate that the kinetics of protein production in vivo is higher in the SK101 strain containing the Tn21 cassette. Time course of specific fluorescence intensity from pGFPuv plasmids transformed in BL21 and SK101 strains shows proteins are produced at a faster rate in the recombinant strain SK101 (see FIG. 6). The experiment demonstrates that the rate of synthesis of recombinant proteins in a system with the gene-cassette also is faster than that in the conventional strain.

### Example VI

### Enhanced Expression Induced by a 792 Bp Fragment, the aadA1 Gene

A 2.4 Kb gene-cassette, obtained from the transposon Tn21 (see SEQ ID NO. 1), and a 792 bp fragment (the aadC1 gene, SEQ ID NO: 2) of the 2.4 Kb gene-cassette, were inserted in the yjaG gene of E. coli K12 strains (MG1655) and E. coli B strain (BL21). The 792 bp fragment was also inserted in the galR gene of E. coli B strain (BL21). E. coli cells were grown till mid-log phase (OD_{600 nm} 0.5) and total proteins were extracted from the cells. Proteins from cultures of equal OD were loaded on gels. Extracts from equal amount of bacterial cells were analyzed for their total protein content. The total cellular protein profiles of strains containing the entire 2.4 kb gene-cassette (SK100) (Lane 3 from left) inserted in the yjaG gene of E. coli chromosome, the 792 bp aadA1 gene (SK102) (Lane 4 from left) inserted in the yjaG gene of E. coli chromosome, the 792 bp aadA1 gene inserted in the galR gene of E. coli chromosome (SK103) (Lane 5 from left), the wild-type strain (MG1655) (Lane 2 from left) are shown in FIG. 7. The total cellular protein content was higher in the strains containing the 2.4 kb cassette or the 792 bp aadA1 gene (strains SK100, SK102 and SK103) than that of the wild-type (WT) strain (see FIG. 8). Again, the strain containing the entire 2.4 kb cassette (SK100) produced a slightly higher protein content than the strain containing only the aadA1 gene (SK102 and SK103) (see FIG. 8). The results indicate that the position insertion of the gene-cassette (for example, the 792 bp aadA1 gene) on the chromosome of the host cell (for example, BL21 derivative SK102 or SK103) can vary. For example, strains SK102 and SK103 harboring the gene-cassette in the yjaG gene or the galR gene of the host chromosome yielded about the same amount of cellular protein content (see FIG. 8).

It is to be understood that the description, specific examples and data, while indicating exemplary embodiments, are given by way of illustration and are not intended to limit the present invention. Various changes and modifications within the present invention will become apparent to the skilled artisan from the discussion, disclosure and data contained herein, and thus are considered part of the invention.

1. A method of enhancing the expression of a protein comprising:
(a) transferring a gene-cassette into a host cell, wherein the gene-cassette comprises a polynucleotide at least 90% identical to the nucleic acid set forth in SEQ ID NO: 1, a polynucleotide at least 90% identical to the nucleic acid set forth in SEQ ID NO: 2, or both a polynucleotide at least 90% identical to the nucleic acid sequence set forth in SEQ ID NO: 1 and a polynucleotide at least 90% identical to the nucleic acid sequence set forth in SEQ ID NO: 2; and
(b) culturing the cell under a suitable growth condition, thereby allowing production and/or accumulation of the protein.

2. A method of reconstructing a host cell for production of a recombinant protein comprising:
(a) transferring a gene-cassette into the host cell, wherein the gene-cassette comprises a polynucleotide comprising the nucleic acid sequence set forth in SEQ ID NO: 1, a polynucleotide comprising the nucleic acid sequence set forth in and/or SEQ ID NO: 2, or a polynucleotide comprising the nucleic acid sequence set forth in SEQ ID NO: 1 and the nucleic acid sequence set forth as SEQ ID NO: 2;
(b) introducing a vector containing a recombinant gene for expression of the recombinant protein; and
(c) culturing the host cell under a suitable growth condition, thereby allowing the production and/or accumulation of the recombinant protein.

3. The method of claim 1, further comprising:
(b) introducing a host cell a vector containing a recombinant gene encoding recombinant protein; and
thereby enhancing expression of the recombinant protein.

4. The method according to claim 1, wherein the host cell is a prokaryotic cell.

5. The method according to claim 1, wherein the host cell is an E. coli.

6. (canceled)

7. The method according to claim 5, wherein the gene-cassette is inserted into yjaG gene or galR gene of the host cell.

8. The method according to claim 1, wherein the host cell is an eukaryotic cell.

9. (canceled)

10. The method according to claim 1, wherein the gene-cassette confers aminoglycoside antibiotic resistance, spectinomycin resistance or streptomycin resistance to the host cell.

11-12. (canceled)

13. The method according to claim 1, wherein the gene-cassette enhances the protein production, protein accumulation, or both, by about 5 to about 200 fold.

14. The method according to claim 3, wherein the gene-cassette is not physically or structurally linked to the vector.

15. The method according to claim 3, wherein the vector is a plasmid, a cosmid, a bacteriophage, or a virus.

16. The method according to claim 1, wherein the gene-cassette comprises a polynucleotide sequence at least 95% identical to the nucleic acid sequence set forth in SEQ ID NO: 1 or SEQ ID NO: 2.

17. The method according to claim 1, wherein the gene-cassette comprises a polynucleotide having the nucleic acid sequence set forth in SEQ ID NO: 1 or SEQ ID NO: 2.

18. The method according to claim 1, wherein the gene-cassette comprises a polynucleotide encoding a polypeptide comprising the amino acid sequence set forth in SEQ ID NO: 3.

19. The method according to claim 1, wherein the gene-cassette comprises a polynucleotide encoding a polypeptide having comprising an amino acid sequence at least 90% sequence identity to the amino acid sequence set forth in SEQ ID NO: 3.

20. A gene-cassette for enhanced production of a protein, wherein the gene-cassette comprises a polynucleotide comprising a nucleic acid sequence at least 90% identical to the nucleic acid sequence set forth in SEQ ID NO: 1 or SEQ ID NO: 2.

21. The gene-cassette according to claim 20, wherein the gene-cassette comprises a polynucleotide comprising a nucleic acid sequence at least 95% sequence identity to the nucleic acid sequence set forth in SEQ ID NO: 1 or SEQ ID NO: 2.

22. The gene-cassette according to claim 20, wherein the gene-cassette comprises a polynucleotide comprising the nucleic acid sequence set forth in SEQ ID NO: 1 or SEQ ID NO: 2.

23. The gene-cassette according to claim 20, wherein the gene-cassette comprises a polynucleotide encoding a polypeptide comprising the amino acid sequence set forth in SEQ ID NO: 3.

24. The gene-cassette according to claim 20, wherein the gene-cassette comprises a polynucleotide encoding a polypeptide comprising an amino acid sequence least 90% identical to the amino acid sequence set forth in SEQ ID NO: 3.

25. The gene-cassette according to claim 20, wherein the cassette confers aminoglycoside antibiotic resistance, spectinomycin resistance or streptomycin resistance.

26-27. (canceled)

28. The gene-cassette according to claim 20, wherein the cassette enhances the protein production and/or accumulation by about 5 to about 200 fold.

29. A host cell comprising the gene-cassette of claim 20.

30. The host cell according to claim 29, wherein the host cell is a prokaryotic cell.

31. The host cell according to claim 30, wherein the host cell is an E. coli.

32-34. (canceled)
